(19) United States
(12) Patent Application Publication
(10) Pub. No.: US 2026/0330251 A1
(43) Pub. Date: Oct. 22, 2026

(54) KNOWLEDGE BASE INFERENCE METHOD AND SYSTEM WITH IMPROVED TRANSPARENCY PROPERTIES

(71) Applicant: NEC Laboratories Europe GmbH, Heidelberg (DE)

(72) Inventors: Zhao XU, Heidelberg (DE); Carolin LAWRENCE, Heidelberg (DE)

(21) Appl. No.: 19/104,722

(22) PCT Filed: Aug. 11, 2023
(86) PCT No.: PCT/EP2023/072347

§ 371 Date: Feb. 19, 2025

(30) Foreign Application Priority Data
Jan. 31, 2023 (EP) 23154282

(51) Int. Cl. G06N 5/022

(52) U.S. Cl. CPC G06N 5/022

(57) ABSTRACT

A computer-implemented method of improving transparency of a knowledge base inference system includes using triples of a knowledge base of facts as training data to train a black-box artificial intelligence (AI) system to predict an original predictive probability of an input triple being true. Each triple specifies a subject entity, an object entity, and a predicate between the subject entity and the object entity. The method also includes using a trained model of the black-box AI system to determine one or more predictive paths that support the prediction of the input triple and/or one or more predictive paths that oppose the prediction of the input triple; and providing, as an output, the original predictive probability of the input triple being true together with the determined one or more predictive paths or an information derived from the one or more predictive paths as explanation for the prediction.

### CROSS REFERENCE TO RELATED APPLICATIONS

This application is a U.S. National Phase application under 35 U.S.C. § 371 of International Application No. PCT/EP2023/072347, filed on Aug. 11, 2023, and claims benefit to European Patent Application No. 23154282.0, filed on Jan. 31, 2023. The International Application was published in English on Aug. 8, 2024 as WO 2024/160388 A1 under PCT Article 21(2).

### FIELD

The present invention relates to a method and system for of improving transparency of a knowledge (KB) inference system.

### BACKGROUND

According to the Artificial Intelligence (AI) act and General Data Protection Regulation (GDPR) in the EU, transparency is an essential requirement for usage of AI technologies, especially in high-risk areas. Knowledge Base Inference (KBI) is a family of important AI technologies and has been successfully applied to many domains with complicated entities and relations, such as digital health that is a high-risk area due to direct influence on life and health of humans. A typical example of a high-risk application is drug development. In the context of a drug development project, a pharmaceutical company must find the side effect of their new drug with existing drugs if patients take them at the same time. In addition, to optimize the revenue or to treat some emerging diseases, such as COVID, the pharmaceutical company may look for possible new uses of their developed drugs beyond the original scope. Namely, a development may aim to learn if a drug/agent ‘a’ can be used to treat a disease ‘d’, <drug a, treat, ?disease d>.

Though KBI systems can give predictions, what is more important for the pharmaceutical companies is to know the reasons of the predictions, such that they facilitate to (1) verify whether the predictions make sense based on existing knowledge, (2) provide hints and prioritize physical experiments that are often expensive and time consuming.

Existing techniques learn as explanations either a large set of single neighbour facts (for reference, see, e.g., Carolin Lawrence, Timo Sztyler and Mathias Niepert: “Explaining Neural Matrix Factorization with Gradient Rollback”, in: Proceedings of AAAI, 2021, https://arxiv. org/abs/2010.05516), or complicated subgraphs (for reference, see, e.g., Hao Yuan et al. “On Explainability of Graph Neural Networks via Subgraph Explorations”, in: Proceedings of the 38^{th} International Conference on Machine Learning, 2021, https://arxiv.org/pdf/2102.05152.pdf), which are often difficult for humans to understand, especially users without AI background. Not only do such implausible explanations fail to improve the trust of the users in the AI systems, in a contrary manner, they amplify users' doubt and hesitation, as acknowledged, e.g., in Himabindu Lakkaraju and Osbert Bastani: ““How do I fool you?”: Manipulating user trust via misleading black box explanations”, in Proceedings of the AAAI/ACM Conference on AI, Ethics, and Society, 2020, https://arxiv.org/pdf/1911.06473.pdf).

### SUMMARY

In an embodiment, the present disclosure provides a computer-implemented method of improving transparency of a knowledge base inference system. The method includes using triples of a knowledge base of facts as training data to train a black-box artificial intelligence (AI) system to predict an original predictive probability of an input triple being true. Each triple specifies a subject entity, an object entity, and a predicate between the subject entity and the object entity. The method also includes using a trained model of the black-box AI system to determine one or more predictive paths that support the prediction of the input triple and/or one or more predictive paths that oppose the prediction of the input triple; and providing, as an output, the original predictive probability of the input triple being true together with the determined one or more predictive paths or an information derived from the one or more predictive paths as explanation for the prediction.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is an overview diagram of a transparency booster engine in accordance with an embodiment of the present disclosure, configured to output predictive paths and suspicious unknown facts to decide correctness of predictions of a black-box AI system;

FIG. 2 is a diagram showing a knowledge graph and predictive paths extracted therefrom according to embodiments of the present disclosure;

FIG. 3 is a diagram showing a process of extracting a predictive path from a drug knowledge graph according to an embodiment of the present disclosure;

FIG. 4 is a diagram showing an operation of a suspicious fact identifier according to an embodiment of the present disclosure;

FIG. 5 is a diagram showing an example suspicious unknown fact that can construct strong a predictive path for a prediction according to an embodiment of the present disclosure; and

FIG. 6 shows a schematic diagram of an example of an apparatus for performing at least one of the methods described herein.

### DETAILED DESCRIPTION

Embodiments of the present disclosure provide to improve the KB inference systems by addressing the technical problem of lack of transparency and lack of trust and explanations by providing increased transparency. This is an especially advantageous improvement for the existing AI systems in high-risk areas for generating correct and human-understandable explanations to the AI-driven predictions; otherwise, these AI systems would not be safe and could not be trusted or deployed in the EU in the near future due to the EU regulations or other countries that may have similar requirements.

In accordance with an embodiment of the present disclosure, the aforementioned desire is addressed by a computer-implemented method of improving transparency of a KB inference system, the method comprising: using triples of a knowledge base of facts as training data to train a black-box AI system to predict a probability-original predictive probability-of an input triple being true, wherein each triple specifies a subject entity, an object entity, and a predicate between the subject entity and the object entity; using the trained model of the black-box AI system to determine one or more predictive paths that support the prediction of the input triple and/or one or more predictive paths that oppose the prediction of the input triple; and providing, as an output, the original predictive probability of the input triple being true together with the determined one or more predictive paths or an information derived therefrom as explanation for the prediction.

The method may be performed by an apparatus, which may be a computer system, or which may be part of a computer system. Accordingly, an aspect of the present disclosure relates to an apparatus comprising one or more processors and one or more storage devices. The apparatus is configured to perform the above method. Another aspect relates to a corresponding computer program comprising instructions which, when the program is executed by a computer, cause the computer to carry out the above method. Another aspect relates to a corresponding non-transitory computer-readable medium comprising instructions which, when executed by a computer, cause the computer to carry out the above method.

According to embodiments of the present disclosure, the afore-mentioned problems are addressed by providing methods and systems that extract (1) supportive predictive paths, (2) contradictory predictive paths, and (3) suspicious unknown facts that can construct strong predictive paths to support or oppose a prediction. For example, for a prediction of a drug ‘a’ targeting a new use of a disease ‘d’, the system disclosed herein may be configured to extract a set of predictive paths from drug knowledge base as explanations, such as <drug a, target, protein p>, <protein p, interact, protein q>, <protein q, pathway, disease d>.

Compared with existing XAI approaches, the methods and systems disclosed herein offer distinguishing properties. In particular, the predictive paths, learned by the system according to the concepts of the present disclosure, match the reasoning processes of humans, and thus can properly solve the limitations of the existing XAI methods in an effective manner. Moreover, the detected suspicious unknown facts screen out the most important unobserved features or relations of entities to test for drug repurposing and drug-drug side effect detection.

In an embodiment, the present disclosure provides a system to improve transparency of knowledge base inference system, wherein the input preparation for the system may include an ML based knowledge base inference system is present which includes facts to improve transparency. The system may be configured to detect, given a specific prediction of the knowledge base inference system, supportive paths of the prediction with importance scores (by running a supportive predictive path generator), contradictory paths of the prediction with importance scores (by running a contradictory predictive path generator) and suspicious facts of the prediction (by running a suspicious unknown fact identifier). The suspicious unknown fact identifier may be configured to screen out important unknown facts that can construct strong predictive paths for the prediction. Accordingly, as output, the system may provide supportive predictive paths, contradictory predictive paths, and suspicious unknown facts.

As such, embodiments of the present disclosure generate human understandable explanations based on instances, attributes and graph structure, and enhance the understandability by learning predictive paths. This simulates the process of how a human learns to make decisions and explains them. Most of existing XAI systems focus on single triple explanations and subgraph explanations, which have been demonstrated by the literature to be difficult to understand. In contrast, the embodiments of the present disclosure provide predictive paths to explain the predictions, which are human-centric, similar as reasoning procedure of a human for the unknown, and thus easy to understand by users. According to embodiments, the method provides an effective way to screen out suspicious unknown facts for end users, such that the end users know what to do to further test the correctness of the predictions.

According to an embodiment, it may be provided that the paths between the subject entity and the object entity of the input triple are extracted from the knowledge base and that a new predictive probability is computed for the input triple for each path, wherein the calculation is based on the assumption that the respective path does not exist in the knowledge base. Each path extracted from the knowledge base may be assigned an importance score based on the respective difference between the original predictive probability and the new predicative probability.

According to an embodiment, it may be provided that a number of paths with highest importance scores, where the new predictive probability is smaller than the original predictive probability, is selected to be outputted as predictive paths that support the prediction of the input triple. Alternatively or additionally, it may be provided that a number of paths with highest importance scores, where the new predictive probability is larger than the original predictive probability, is selected to be outputted as predictive paths that oppose the prediction of the input triple.

According to an embodiment, it may be provided that the length of the paths that are extracted from the knowledge base is configurable by a user of the KB inference system.

For instance, for the case of an extracted path of length L=1, the extracted path consists of one training triple of the knowledge base. In this case, the new predictive probability for the input triple may be calculated as follows: first, the embedding vectors of the subject entity and the object entity, respectively, of the input triple from the trained model are updated with the scaled gradients of the subject entity and the object entity, respectively, of said one training triple corresponding to the respective path. Then, the trained black-box AI system with the updated embedding vectors is used as input to infer the new predictive probability for the input triple.

For the case of an extracted path of length L=2, the extracted path consists of a first training triple of the knowledge base that is linked with the subject entity of the input triple and a second training triple of the knowledge base that is linked with the object entity of the input triple, the first and the second training triple being linked via the same object entity. In this case, the new predictive probability for the input triple may be calculated as follows: first, the embedding vector of the subject entity of the input triple from the trained model is updated with the scaled gradient of the subject entity of said first training triple, and the embedding vector of the object entity of the input triple from the trained model is updated with the scaled gradient of the object entity of said second training triple. Then, the trained black-box AI system with the updated embedding vectors is used as input to infer the new predictive probability for the input triple.

For the case of an extracted path of length L=3, the extracted path consists of a first training triple of the knowledge base that is linked with the subject entity of the input triple, a third training triple of the knowledge base that is linked with the object entity of the input triple, and a second training triple of the knowledge base that links the first and the third training triples. In this case, the influence of said second training triple on the embedding vectors of the subject entity and the object entity of the input triple may be taken into account by updating the embedding vector of the subject entity of said second training triple from the trained model with the scaled gradient of the subject entity of said second training triple.

For the case of an extracted path of length L>3, i.e. L=n with n>3, the new predictive probability can be computed equivalently. In this case, the extracted path consists of a first training triple of the knowledge base that is linked with the subject entity of the input triple, an n'th training triple of the knowledge base that is linked with the object entity of the input triple, and a number (i.e., n-2) of middle training triples of the knowledge base that link the first and the n'th training triples. The influence of said middle training triples on the embedding vectors of the subject entity and the object entity of the input triple may be taken into account by updating the embedding vectors of the subject entities of said middle training triples from the trained model with the scaled gradients of the subject entities of said middle training triples.

According to an embodiment, it may be provided that sensitive entities of an input triple are identified, wherein the sensitive entities include the subject entity and the object entity of the input triple as well as the n-order neighbours of the subject entity and the object entity in the knowledge base, with n being a predefined or configurable parameter. The probabilities of the unknown triples between all identified sensitive entities may be predicted by using the black-box AI system.

According to an embodiment, it may be provided that the unknown triples between all identified sensitive entities are added as training triples into the knowledge base. A graph search method may be used to identify paths between the subject entity and the object entity of the input triple. From the identified paths, those paths that include unknown triples between sensitive entities may be selected and scored. Based on the scoring, number of top-scored paths may be filtered out and the unknown triples in the paths filtered out may be selected as suspicious unknown facts to be tested for validation of the prediction.

According to an embodiment, the knowledge base of facts may be a knowledge base of drugs, diseases, proteins and related information. Triples of the knowledge base of facts may be used as training data to train the black-box AI system to predict a probability of a given drug targeting a given disease.

According to an embodiment, the knowledge base of facts may be a knowledge base of drugs, diseases, proteins and related information. Triples of the knowledge base of facts may be used as training data to train the black-box AI system to predict a probability of a given first drug interacting with a given second drug.

According to an embodiment, the knowledge base of facts may be a knowledge base of a city, including information of districts, schools, bus/subway lines, stores, entertainment and possibly further related information. Triples of the knowledge base of facts may be used as training data to train the black-box AI system to predict a probability of a given action satisfying a given target.

Embodiments of the present disclosure provide a solution to improve transparency of knowledge base inference systems via automatically learning: (1) supportive predictive paths, (2) contradictory predictive paths, and (3) suspicious unknown facts that can construct strong predictive paths to support or oppose the predictions. As such, methods and systems according to embodiments of the present disclosure are capable of delivering human understandable explanations of why knowledge base inference systems make certain predictions. Embodiments of the present disclosure have a variety of application areas, such as drug repurposing or drug-drug side effect detection in digital health.

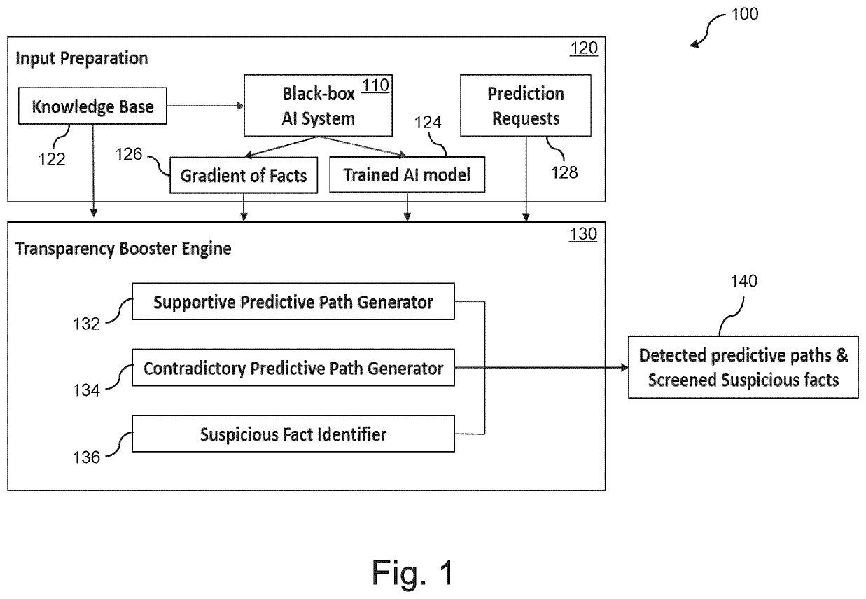

FIG. 1 schematically illustrates a system 100 to improve transparency of a black-box AI system 110 for knowledge base inference in accordance with an embodiment of the present disclosure. For easier understanding of the concepts proposed herein, the mode of operation of the system 100 will be introduced based on a running example of drug development, which aims to re-target drugs, i.e., to find new usage(s) of an existing drug beyond its original scope. However, the chosen application scenario is to be understand to be merely exemplarily and, as will be appreciated by those skilled in the art, the proposed concepts can be applied in many different contexts and applications.

With reference to FIG. 1, assume that there is a drug a. The black-box AI system 110 predicts <drug a, treat, disease d> with probability P. Embodiments of the present disclosure provide methods and systems that extract predictive paths with important scores, which support or oppose the prediction of the black-box AI system 110. Furthermore, embodiments of the present disclosure provide methods and systems that—additionally or alternatively—identify suspicious unknown facts, by knowing which strong predictive paths can be constructed to support or oppose the predictions.

As shown in FIG. 1, the system 100 to improve transparency of the existing black-box AI system 110 includes an input preparation component 120 and a transparency booster engine 130, which is the actual component configured to improve transparency and which provides detected predictive paths and/or screened suspicious facts as output 140, as will be described in detail below.

### Input Preparation

According to an embodiment, the system 100 includes the following inputs:
Knowledge base: The knowledge base 122 includes a (large) set of triples, which play as training data. Each triple is of format <subject, predicate, object>, e.g., <protein q, pathway, disease d>, where subject and object are entities that can be visualized as nodes in the knowledge graph, predicate specifies a relation between subject and object that can be viewed as a label of an edge between the two nodes.
Black box AI system: The Black box AI system 100 is a knowledge base inference (KBI) system, e.g., a KBlrn framework, as described in A. Garcia-Duran and M. Niepert: “KBLRN: End-to-End Learning of Knowledge Base Representations with Latent, Relational, and Numerical Features”, arXiv:1709.04676, which is hereby incorporated herein by reference. The Black box AI system 100 provides predictions without explanations of how the predictions are achieved. Based on the AI regulations, there is a desire to improve its transparency, i.e., generating explanations for each prediction.
Training is to train the black-box AI system 100 with the triples in the knowledge base 122 as training data in order to obtain a trained AI model 124 that can be used for predictions. During the training procedure, the scaled gradients of each training triple may be recorded. The scaled gradient is multiplication of the learning rate and the gradient. The gradient is computed as the partial derivative of the loss of a training triple with respect to the embedding vector of each involved entity. For instance, to get the gradients efficiently, an existing method called Gradient Rollback may be employed, as described in WO 2021/244766 A1, which is hereby incorporated by reference herein. The gradient specifies to what extent the training triple contributes to the embedding vector of the entity. The larger the gradient is, the more important the training triple is for the entity. The recorded scaled gradients may be stored in a database, denoted gradient of facts 126 in FIG. 1.
Predictions: After the black-box AI system 110 is trained, the trained model 124 can be used to make prediction requests 128 and to obtain predictions in response. The predictions are represented as probabilities of unknown triples are true. For example, a user of the system may want to know if drug a can be used to treat disease d. Accordingly, the predicted triple <drug a, treat, disease d> may be provided as input triple to the AI system 110, which then provides the probability P of the triple being true.

In this embodiment, the dataflow as shown in FIG. 1 is as follows. The knowledge base 122, formatted as a set of triples, is inputted into the black box AI system 110 to train a model, i.e. to obtain trained AI model 124. During the training, the scaled gradients 126, represented as vectors, are saved for each triple. The trained model 124, the knowledge base 122, and the gradients 126 of each triple in the knowledge base 122 are inputs into the proposed transparency booster engine 130. During prediction, i.e., when a user requests a predicted triple, formatted as, e.g., <subject, predicate, object>, the black box AI system 110 uses the trained model 124 to compute a probability of the triple being true, and the proposed XAI system 100 provides as output 140 explanations for the prediction according to the inputs.

Supportive and Contradictory Predictive Path Generators 132, 134

As illustrated in the embodiment of FIG. 1, the transparency booster engine 130 comprises a supportive predictive path generator 132 and a contradictory predictive path generator 134, the operation of which is described in more detail below.

For each triple t*=<s*, t*, o*> predicted by the black box AI system 110, with s* as subject, o* as object and r* as predicate, the generators 132, 134 may be configured to extract predictive paths with importance scores. The supportive generator 132 outputs predictive paths that support a prediction, i.e., if a respective path were not existent in the training data, the predictive probability of the triple t* would decrease. In contrast, the contradictory generator 134 outputs predictive paths that oppose the prediction, i.e., without a respective path, the predictive probability would increase. The importance score of a path is proportional to the probability change. The larger the change of the predictive probability after removing the path, the higher is the importance of the path for the respective prediction.

In particular, for a predicted triple t*=<s*, t*, o*>, it may be provided that first the paths between s* and o* are extracted from the knowledge base 122. For this purpose, any off-the-shelf graph search method can be employed, such as Depth-First Search (DFS) and Breadth-First Search (BFS). The paths can be of length L=1, 2, 3 . . . which may be a pre-installed parameter or which may be selected by the user of the system according to the specific requirements. Then, for each existing path extracted from the knowledge base 122, a new predictive probability may be computed for the predicted triple t* if the respective path is removed from the knowledge base 122. In other words, it is calculated, for each path extracted from the knowledge base 122 in relation to the predicted triple t*, what the predictive probability for the triple t* would be, if the respective path didn't exist in the training data.

For a path of length L=1 (i.e., a training triple t with s* as subject/object, o* as object/subject, and r as predicate, r≠r*), the predictive generators 132, 134 may be configured to compute p(t*=T|t=F), where T and F are shortens of True and False, respectively. Accordingly, the above expression denotes the probability of the predicted triple t* being true under the condition that the training triple t (with predicate r≠r*) is false (corresponding to the non-existence of the respective path).

According to an embodiment of the present disclosure, the above probability may be calculated based on the scaled gradient of s* and o* of the training triple t. To this end, first, the scaled gradient of s* and o* of the training triple t may be looked up from the input data, and then the embedding vectors of s* and o* from the trained AI model 124 (denoted xₛ*, and xₒ* herein) may be updated as follows:

xₛ*_{_updated}=xₛ*+δ(s*,t);xₒ*_{_updated}=xₒ*+δ(o*,t)

where δ(s*, t) and δ(o*, t) denote the scaled gradient of s* and o*, respectively, of the training triple t. The updated embedding vectors may then be used as input to the black-box KBI system 110 to compute the new probability of the predicted triple t*. If the new probability is smaller than the original probability, then the respective path is a supportive predictive path, otherwise it is a contradictory one. The importance score of the path can be computed based on the difference of the new and original probabilities. For instance, the importance score may be calculated as f(Δₚ), where Δₚ denotes the difference of the new and original probabilities, and where f(·) can be any monotonically increasing function.

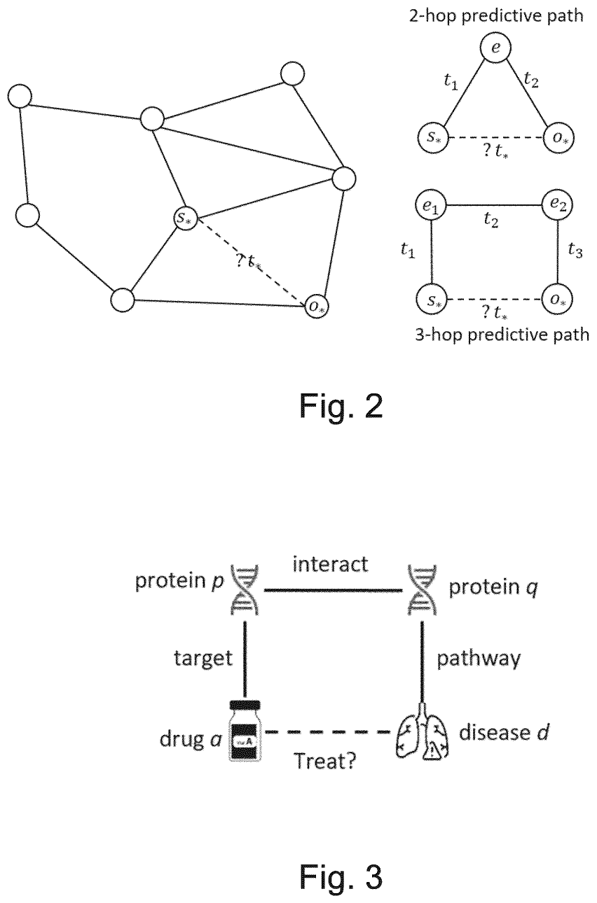

Considering a path of length L=2, the path consists of two training triples, namely, as shown in FIG. 2, t₁ linking with the subject s* and t₂ linking with the object o*, wherein the two triples t₁ and t₂ are linked with an arbitrary entity e. For this case, the predictive generators 132, 134 may be configured to compute p(t*=T|t₁=F, t₂=F), i.e. the probability of the predicted triple t* being true under the condition that the training triples t₁ and t₂ are false (corresponding to the non-existence of the respective path).

According to an embodiment of the present disclosure, the above probability may be calculated based on the scaled gradient of s* of the training triple t₁ and the scaled gradient o* of the training triple t₂. To this end, first, the scaled gradient of s* of the training triple t₁ and the one of o* of the training triple t₂ may be obtained from the input data. Then, the embedding vectors of s* and o* may be updated according to the above equation, i.e. by computing

xₛ*_{_updated}=xₛ*+δ(s*,t₁);xₒ*_{_updated}=xₒ*+δ(o*,t₂)

where δ(s*, t₁) denotes the scaled gradient of s* of training triple t₁ and δ(o*, t₂) denotes the scaled gradient of o* of the training triple t₂.

Again, the updated embedding vectors of s* and o* may be used as input to the black-box KBI system 110 to compute the new probability of the predicted triple t*. If the new probability is smaller than the original probability, then the respective path is a supportive predictive path, otherwise it is a contradictory one.

Considering a path of length L=3, the path consists of three training triples, namely, as shown in FIG. 2, t₁ linking with the subject s*, t₃ linking with the object o*, and t₂ linking the two triples t₁ and t₃. The challenge in this case is how to quantify the influence of t₂. As the triple t₂ does not directly link to the subject s* and the object o*, it is not possible to calculate the new embedding vectors like in the cases of the short paths (L≤2) as described above. However, the training triple t₂ indeed influences the embedding of s* and o*, as it changes the points where the gradients of s* and o* are computed. To compute the influence of the entire path, embodiments of the present disclosure propose a probabilistic approach, which can easily be extended to compute the influence of longer paths with L>3. In particular, according to an embodiment, the predictive generators 132, 134 may be configured to compute the probability p(t*=T, t₁=F, t₂=F, t₃=F), i.e. the probability of the predicted triple t, being true under the condition that the training triples t₁, t₂ and t₃ are false (corresponding to the non-existence of the respective path).

The probability can be decomposed based on the chain rule in probability theory:

p(t*=T,t₁=F,t₂=F,t₃=F)

≈p(t*=T|t₁=F,t₃=F)p(t₁=F|t₂=F)p(t₃=F|t₂=F)p(t₂=F)

The first term p(t*=T|t₁=F, t₃=F) can be computed as the above way (i.e., like in the case of a path of length L=2).

The second term p(t₁=F|t₂=F) may be computed as follows: First, the scaled gradient of the entity e₁ of the training triple t₂ is determined, then the embedding vector of e₁ is updated as

xₑ_{₁}_{updated}=xₑ_{₁}+δ(e₁,t₂)

Next, the updated embedding is used as input to the black-box KBI system 110, and the probability of the triple t₁ is computed, which is p(t₁=F|t₂=F).

The third term p(t₃=F|t₂=F) can be computed in a similar way as the second term.

The last term p(t₂=F) can directly be computed with the black-box KBI system 110.

For a path of length L>3, the new probability of the predicted triple t, may be computed equivalently as described above for a path of length L=3.

### Suspicious Unknown Fact Identifier 136

This component, which may be also implemented as part of the transparency boost engine 130, aims to screen out unknown triples, by knowing which strong predictive paths can be constructed to support or oppose a given prediction.

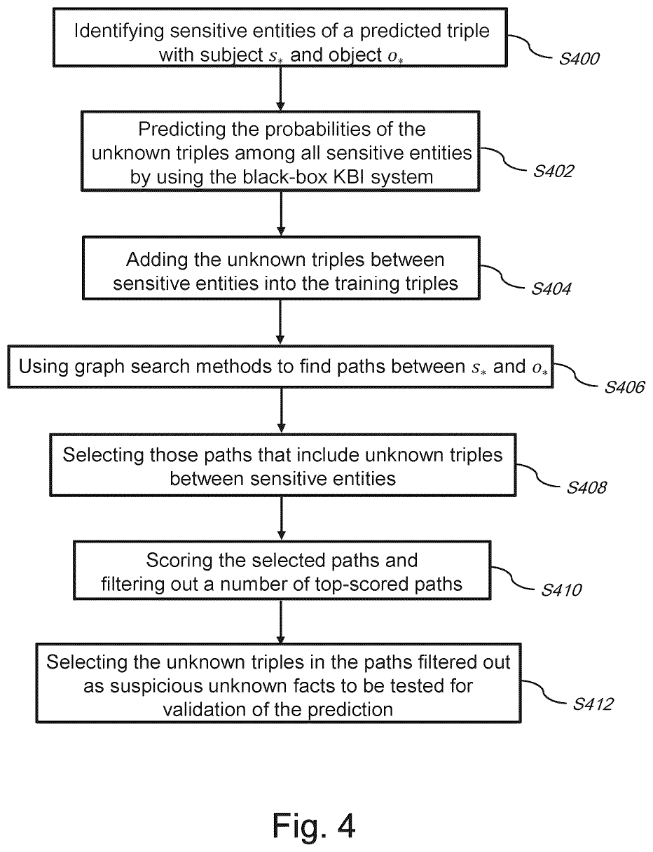

According to an embodiment of the present disclosure, illustrated in FIG. 4, the identifier 136 may be configured to first identify the sensitive entities of the predicted triple (as shown at step S400 in FIG. 4). The sensitive entities include the subject and object entities of the predicted triple, i.e. s* and o*, as well as the n-order neighbors of s* and o* in the training knowledge base 122. The parameter n may be a pre-installed parameter or may be configurable to be specified by the user of the system according to the specific requirements. In an embodiment, it may be provided that a user can also add extra entities as sensitive ones, or remove some ones from the set.

Given the specified set of sensitive entities, in a next step (shown at step S402), the black-box KBI system 110 may be used to predict the probabilities of the unknown triples among all sensitive entities. Next (shown at step S404), these triples involving sensitive entities are added to the training triples, and graph search methods are applied (step S406) to find paths between s* and o*, i.e. the subject and object entities of the predicted triple.

According to the illustrated embodiment, given the set of found paths between s* and o*, it may be provided to select those paths from the set that include unknown triples between sensitive entities (as shown at step S408). In a next step, shown at step S410, these selected paths may be scored, in accordance with the methods described above for different path lengths in connection with the operation of the predictive graph generators 132, 134. Then, a filter may be applied that may be configured to filter out the K top-scored paths or the paths having a score that exceed a given score threshold. Both the parameter K and the score threshold may be pre-installed parameters or may be configurable to be specified by the user of the system according to the specific requirements.

According to an embodiment and as shown at step S412, the identifier 136 may be configured to select the unknown triples in the paths that were filtered out, and to provide the selected unknown triples as suspicious unknown facts in the output 140. These unknown triples can then be specifically tested for validation of the prediction.

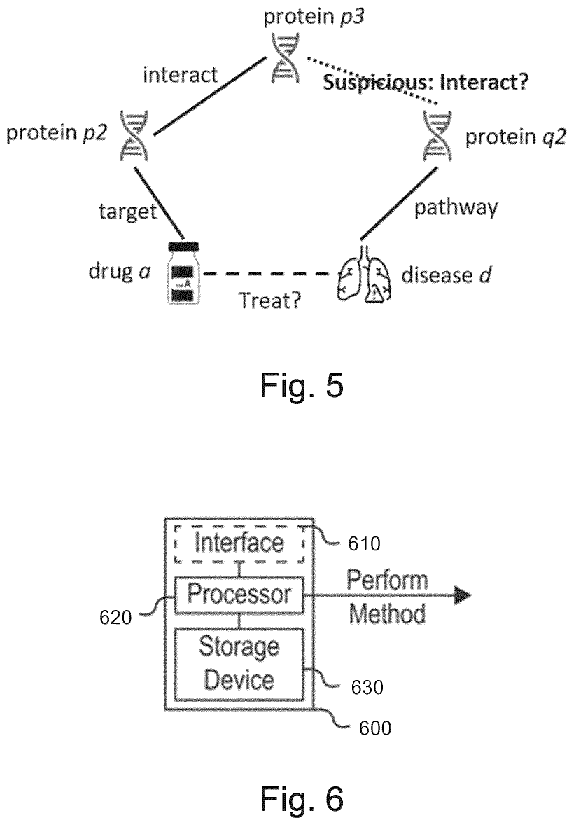

FIG. 5 illustrates an example of suspicious unknown fact identification in a drug knowledge graph. That prediction considered in the illustrated example is the prediction <drug a, treat, disease d>. In the illustrated example it is assumed that step S400 described above in connection with FIG. 4 revealed, including but not limited to, the entities ‘protein p2’, ‘protein p3’, and ‘protein q2’ as sensitive entities of the predicted triple in question.

Furthermore, it is assumed that step S406 described above revealed a number of paths between the subject of the prediction (i.e. ‘drug a’) and the object of the prediction (i.e. ‘disease d’), including the path exemplarily shown in FIG. 5. The selection made in step S408 would include the path shown in FIG. 5, as it contains an unknown triple between sensitive entities, namely the triple <protein p3, interact?, protein q2>. Consequently, this path would take part in the scoring executed in step S410 and would be filtered out in case of receiving a top score. In this case, in step S412, the unknown triple <protein p3, interact?, protein q2> with the BS selected as suspicious unknown fact, which could be tested separately for validation of the prediction. If it turns out that ‘protein p3’ actually interacts with ‘protein q2’, this would support the prediction that ‘drug a’ actually treats ‘disease d’.

FIG. 6 shows a schematic diagram of an example of an apparatus 600 for performing at least one of the methods described herein. The apparatus 600 comprises one or more processors 620 and one or more storage devices 630. Optionally, the apparatus further comprises one or more interfaces 610. For example, the apparatus 600 may be a computer system. The one or more processors 620 are coupled with the one or more interfaces 610 and the one or more storage devices 630. For example, the one or more processors 620 may be configured to provide the functionality of the apparatus 600, e.g., in conjunction with the one or more interfaces 610 (for exchanging information with one or more entities outside the apparatus 600) and/or with the one or more storage devices 630 (for storing information, such as machine-readable instructions, a knowledge graph, a machine-learning model, or a link prediction machine-learning model). In various examples, the one or more processors 620 may be configured to execute a plurality of machine-readable instructions of a computer program. In other words, the functionality of the apparatus 600 may be provided by the one or more processors executing the machine-readable instructions of a computer program.

For example, the apparatus 600 may be used to (e.g., configured to, via corresponding machine-readable instructions) perform at least one of the methods described herein, such as the methods illustrated in connection with FIGS. 2 and 3 or the method of FIG. 4.

For example, the interface 610 may include or correspond to a network interface and/or a device interface configured to be communicatively coupled to one or more other devices, such as processor 620 or sensors in a domain of interest. For example, the one or more interfaces 610 may include a transmitter, a receiver, or a combination thereof (e.g., a transceiver), and may enable wired communication, wireless communication, or a combination thereof. The interface 610 may provide information to a user interacting with said interface 610.

For example, the one or more processors 620 may include or correspond to a digital signal processor (DSP), a graphical processing unit (GPU), and/or a central processing unit (CPU). The one or more processors 620 may be coupled to the storage device(s) 630. The storage device(s) 630 may include instructions (e.g., executable instructions), such as computer-readable instructions or processor-readable instructions. The instructions may include one or more instructions that are executable by a computer, such as by each of the one or more processors 620.

For example, the one or more storage devices 630 may include or correspond to non-volatile storage devices, such as magnetic disks, optical disks, or flash memory devices. The storage devices 630 may include both removable and non-removable memory devices. The storage devices 630 may be configured to store an operating system, images of operating systems, applications, and program data. The storage devices 630 may also store the supportive predictive path generator 132, the contradictory predictive path generator 134, the suspicious fact identifier 136, the Knowledge Base 122, the trained model 124 and the Gradient of Fact Database 126. In a particular embodiment, the storage devices 630 include tangible computer-readable media.

Hereinafter, some application scenarios of the methods and systems disclosed herein will be described. As will be appreciated by those skilled in art, the described application scenarios are only exemplary and many other application scenarios in a variety of different technological fields can be realized likewise.

A possible use case of the concepts disclosed herein is in the technical field of drug repurposing. In drug development, repurposing of drugs is an important task to create extra revenue of a pharmaceutical company and treat emerging diseases. Drug repurposing is a process of identifying new uses for approved drugs. For example, some pharmaceutical drugs were re-targeted for COVID in early stage of the pandemic. To avoid expensive physical tests, AI models are employed to computationally screen out the new targets. Biomedical researchers may have a desire to know why an AI model makes a specific prediction, and importantly, they may have a desire to have an XAI system to help screen out the informative clues of why a specific drug can target a specific disease.

In the context of this use case, it may be provided that the data source is a knowledge base of drugs, diseases, proteins, and other related information. Typical predicates between the entities of the respective knowledge graph would include ‘target’, ‘interact’, ‘pathway’, etc. Using triples of such knowledge base as training data, a black-box AI system can be trained to predict a probability that a given drug targets a given disease.

According to an embodiment of the present disclosure, the trained model of the black-box AI system may be used to determine one or more predictive paths that support the prediction of the input triple and/or one or more predictive paths that oppose the prediction of the input triple. In other words, the system may learn predictive paths of a ML-driven prediction. For example, for a prediction of a ‘drug a’ targeting a ‘disease d’, the system may offer a set of predictive paths together with their respective importance scores, such as <drug a, target, protein p>, <protein p, interact, protein q>, <protein q, pathway, disease d>, etc.

Accordingly, embodiments of the present disclosure provide a computer-implemented method of improving transparency of a knowledge base, KB, inference system, the method comprising:
using triples of a knowledge base of drugs, diseases, proteins and possibly further related information as training data to train a black-box AI system to predict a probability—original predictive probability—of a specific drug targeting a specific disease;
using the trained model of the black-box AI system to determine one or more predictive paths that support the prediction of said drug targeting said disease and/or one or more predictive paths that oppose the prediction of said drug targeting said disease; and
providing, as an output, the original predictive probability together with the determined one or more predictive paths or an information derived therefrom as explanation for the prediction.

The predictive paths may be provided as an explanatory output of why a drug is predicted to target a disease. In addition, suspicious unknown facts, generated according to the concepts described herein, may be provided as output to test for demonstration and/or verification of the prediction. Accordingly, the above method may further comprise the step of using the output of one or more predictive paths or an information derived therefrom and/or the output of suspicious unknown facts to conduct tests for demonstration and/or verification of the prediction.

The detected predictive paths identify why the black box AI system predicts the drug can treat the new disease. The identified suspicious unknown information provides the biomedical researchers with more context information about what to validate and investigate for the new usage of the drug, if they are not certain about the prediction. This can accelerate screening and physical testing.

Another possible use case of the concepts disclosed herein is in the technical field of drug side effect detection. During drug development and usage of existing drugs, a complicated but critical medical question is: What happens if a patient takes two pharmaceutical drugs simultaneously. Importantly, pharmaceutical companies and doctors need to know whether two drugs interact with each other to cause side effects potentially. Testing the effect of a drug-drug combination in a physical environment is impractical, since there are many combinations and variables, which makes verification prohibitively expensive and time consuming. A far more efficient process is to screen with an AI model, which makes novel predictions about what occurs when two drugs are taken simultaneously. The biomedical researchers can then focus the physical tests to examine promising combinations. However, the AI model is often a black box that only offers a result. The biomedical researchers may have a desire to know why the AI model makes the prediction, such that their trust is increased, and more importantly, the explanations give clues of how two drugs interact with each other.

In the context of this use case, it may be provided that—like in the previous use case—the data source is a knowledge base of drugs, diseases, proteins, and other related information. Typical predicates between the entities of the respective knowledge graph would include ‘target’, ‘interact’, ‘pathway’, etc. Using triples of such knowledge base as training data, a black-box AI system can be trained to predict a probability that two given drugs interact with each other to cause side effects.

According to an embodiment of the present disclosure, the trained model of the black-box AI system may be used to determine one or more predictive paths that support the prediction of the input triple and/or one or more predictive paths that oppose the prediction of the input triple. In other words, the system may learn to extract predictive paths of how a drug interacts with another drug. For example, for a prediction of a ‘drug a’ interacting with a ‘drug b’, the system may offer a set of predictive paths together with their respective importance scores, such as <drug a, target, protein p>, <protein p, interact, protein q>, <protein q, is targeted by, drug b>, etc.

Accordingly, embodiments of the present disclosure provide a computer-implemented method of improving transparency of a knowledge base, KB, inference system, the method comprising:
using triples of a knowledge base of drugs, diseases, proteins and possibly further related information as training data to train a black-box AI system to predict a probability—original predictive probability—of a specific first drug interacting with a specific second drug;
using the trained model of the black-box AI system to determine one or more predictive paths that support the prediction of said first drug interacting with said second drug and/or one or more predictive paths that oppose the prediction of said first drug interacting with said second drug; and
providing, as an output, the original predictive probability together with the determined one or more predictive paths or an information derived therefrom as explanation for the prediction.

The predictive paths may be provided as an explanatory output of why or how a drug interacts with another drug. In addition, suspicious unknown facts, generated according to the concepts described herein, may be provided as output to test for demonstration and/or verification of the prediction. Accordingly, the above method may further comprise the step of using the output of one or more predictive paths or an information derived therefrom and/or the output of suspicious unknown facts to conduct tests for demonstration and/or verification of the prediction.

The detected predictive paths identify why the black box AI system predicts the two drugs interact with each other. The identified suspicious unknown information provides the biomedical researchers with more context information about what to validate and investigate for the new usage of the drug, if they are not certain about the prediction. This can accelerate screening and physical testing.

Yet another possible use case of the concepts disclosed herein are digital government systems, in particular action checking for policy optimization in digital government system. In a digital government system, the end users, i.e., policy makers of a city, often need to optimize actions of a city to satisfy a specific target, such as favouring inhabitation of the city. Identifying the optimal actions and explaining the reasons of the actions are important for a digital government system, such that the policy makers can know what to do and why. According to embodiments of the present disclosure, the explanations not only explain how the AI-driven system arrives at the results, but also provide hints of important features of the selected actions and reasonability of the actions. Importantly, the AI-driven results must be explained due to the regulations of the EC, otherwise the system can't be deployed and used in the EU.

In the context of this use case, it may be provided that the data source is a knowledge base of a city, including e.g., information of districts, schools, bus/subway lines, stores, and entertainment. Typical predicates between the entities of the respective knowledge graph would include ‘connect’, ‘target’, ‘interact’, ‘attract’, etc. Using triples of such knowledge base as training data, a black-box AI system can be trained to predict a probability that a specific action taken by the policymakers of the city satisfies or meets a specific target.

According to an embodiment of the present disclosure, the trained model of the black-box AI system may be used to determine one or more predictive paths that support the prediction of the input triple and/or one or more predictive paths that oppose the prediction of the input triple. In other words, the system may learn predictive paths of a ML-driven predicted action. For example, for a prediction of a ‘action a’ satisfying a ‘target t’, the system may offer a set of predictive paths together with their respective importance scores, such as <action a, improves, bus line b>, <bus line b, connects with, subway line s>, <subway line s, supports, target t>, etc.

Accordingly, embodiments of the present disclosure provide a computer-implemented method of improving transparency of a knowledge base, KB, inference system, the method comprising:
using triples of a knowledge base of a city including, e.g., information of districts, schools, bus/subway lines, stores, entertainment and possibly further related information as training data to train a black-box AI system to predict a probability—original predictive probability—of a specific action satisfying a specific target;
using the trained model of the black-box AI system to determine one or more predictive paths that support the prediction of said action satisfying said target and/or one or more predictive paths that oppose the prediction of said action satisfying said target; and
providing, as an output, the original predictive probability together with the determined one or more predictive paths or an information derived therefrom as explanation for the prediction.

The predictive paths may be provided as an explanatory output of why an action is predicted to satisfy a target. In addition, suspicious unknown information of the city, generated according to the concepts described herein by knowing which strong predictive paths can be constructed to support or oppose the predicted action, may be provided as output to test for demonstration and/or verification of the prediction. Accordingly, the above method may further comprise the step of using the output of one or more predictive paths or an information derived therefrom and/or the output of suspicious unknown facts to conduct tests for demonstration and/or verification of the prediction.

The detected predictive paths identify why the black box AI system predicts the new actions, by for example identifying why not more people live in a certain area of the city (e.g. not enough schools, therefore the new action is to build a new school). According to the explanations, if the prediction is certain, then the action is added to the to-do-list of the city.

Otherwise, the identified suspicious unknown information provides the end users with more context information about what to validate and investigate for the new actions.

It should be noted that the above example of an <action a, satisfies?, target t> prediction can be in many different context in various technical fields, such as in the context of predictive maintenance applications.

To summarize, the present disclosure provides methods to decide correctness of a new prediction by extracting positive, i.e. supportive, and negative, i.e. contradictory, predictive paths from a knowledge base via gradient based probabilistic inference. If a high number (e.g. above a configurable threshold) of contradictory predictive paths with importance scores that exceed a configurable score threshold are detected, then the new prediction is decided to be false. In addition, the present disclosure provides methods to identify suspicious unknown facts for deciding correctness of a new prediction by constructing positive and contradictory predictive paths with large important scores. This is an enabler to screen promising factors from numerous candidates for expensive and time-consuming tests.

Many modifications and other embodiments of the present disclosure set forth herein will come to mind to the one skilled in the art to which the present disclosure pertains having the benefit of the teachings presented in the foregoing description and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

While subject matter of the present disclosure has been illustrated and described in detail in the drawings and foregoing description, such illustration and description are to be considered illustrative or exemplary and not restrictive. Any statement made herein characterizing the invention or disclosure is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1: A computer-implemented method of improving transparency of a knowledge base inference system, the method comprising:
using triples of a knowledge base of facts as training data to train a black-box artificial intelligence (AI) system to predict an original predictive probability of an input triple being true, wherein each triple specifies a subject entity, an object entity, and a predicate between the subject entity and the object entity;
using a trained model of the black-box AI system to determine one or more predictive paths that support the prediction of the input triple and/or one or more predictive paths that oppose the prediction of the input triple; and
providing, as an output, the original predictive probability of the input triple being true together with the determined one or more predictive paths or an information derived from the one or more predictive paths as explanation for the prediction.

2: The method according to claim 1, further comprising:
extracting, from the knowledge base, paths between the subject entity and the object entity of the input triple; and
computing, for each path, a new predictive probability for the input triple, wherein the computation is based on the assumption that the respective path does not exist in the knowledge base

3: The method according to claim 2, further comprising:
assigning each path extracted from the knowledge base an importance score based on a respective difference between the original predictive probability and the new predicative probability.

4: The method according to claim 3, further comprising:
selecting a number of paths with highest importance scores, where the new predictive probability is smaller than the original predictive probability, to be output as predictive paths that support the prediction of the input triple; and/or
selecting a number of paths with highest importance scores, where the new predictive probability is larger than the original predictive probability, to be output as predictive paths that oppose the prediction of the input triple.

5: The method according to claim 2, wherein, for the case of an extracted path of length L=1, where the extracted path consists of one training triple of the knowledge base the new predictive probability for the input triple is calculated by:
updating embedding vectors of the subject entity and the object entity, respectively, of the input triple from the trained model with scaled gradients of the subject entity and the object entity, respectively, of the one training triple corresponding to the respective path; and
using the trained model of the black-box AI system with the updated embedding vectors as input to infer the new predictive probability for the input triple.

6: The method according to claim 2, wherein, for the case of an extracted path of length L=2, where the extracted path comprises a first training triple of the knowledge base that is linked with the subject entity of the input triple and a second training triple of the knowledge base that is linked with the object entity of the input triple, the first and the second training triple being linked via the same object entity, the new predictive probability for the input triple is calculated by:
updating an embedding vector of the subject entity of the input triple from the trained model of the black-box AI system with a scaled gradient of the subject entity of the first training triple and updating an embedding vector of the object entity of the input triple from the trained model of the black-box AI system with a scaled gradient of the object entity of the second training triple; and
using the trained model of the black-box AI system with the updated embedding vectors of the subject entity and the object entity as input to infer the new predictive probability for the input triple.

7: The method according to claim 2, further comprising:
for the case of an extracted path of length L=3, where the extracted path comprises a first training triple of the knowledge base that is linked with the subject entity of the input triple, a third training triple of the knowledge base that is linked with the object entity of the input triple, and a second training triple of the knowledge base that links the first and the third training triples,
taking an influence of the second training triple on embedding vectors of the subject entity and the object entity of the input triple into account by updating the embedding vector of the subject entity of the second training triple from the trained model of the black-box AI system with a scaled gradient of a subject entity of the second training triple.

8: The method according to claim 1, further comprising:
identifying sensitive entities of an input triple, the sensitive entities comprising the subject entity and the object entity of the input triple as well as n-order neighbours of the subject entity and the object entity in the knowledge base, with n being a predefined or configurable parameter; and
predicting probabilities of unknown triples between all identified sensitive entities by using the trained model of the black-box AI system.

9: The method according to claim 8, further comprising:
adding the unknown triples between all identified sensitive entities as training triples into the knowledge base;
using a graph search method to identify paths between the subject entity and the object entity of the input triple; and
selecting, from the identified paths, those paths that comprise unknown triples between sensitive entities.

10: The method according to claim 9, further comprising:
scoring the selected paths and filtering out a number of top-scored paths; and
selecting the unknown triples in the paths filtered out as suspicious unknown facts to be tested for validation of the prediction.

11: The method according to claim 1, wherein the knowledge base of facts is a knowledge base of drugs, diseases, proteins and related information, and wherein triples of the knowledge base of facts are used as training data to train the black-box AI system to predict a probability of a given drug targeting a given disease.

12: The method according to claim 1, wherein the knowledge base of facts is a knowledge base of drugs, diseases, proteins and related information, and wherein triples of the knowledge base of facts are used as training data to train the black-box AI system to predict a probability of a given first drug interacting with a given second drug.

13: The method according to claim 1, wherein the knowledge base of facts is a knowledge base of a city comprising information of districts, schools, bus/subway lines, stores, and entertainment, and wherein triples of the knowledge base of facts are used as training data to train the black-box AI system to predict a probability of a given action satisfying a given target.

14: An apparatus, the apparatus comprising one or more processors and one or more storage devices, wherein the apparatus is configured to perform the method according to claim 1.

15: A tangible, non-transitory computer readable medium comprising instructions which, when executed by one or more processors, facilitate the performance of the method of claim 1.
